(19) United States
(12) Patent Application Publication
(10) Pub. No.: US 2012/0041424 A1
(43) Pub. Date: Feb. 16, 2012

(54) Connector for Connecting Parts of a Medical Device

(75) Inventors: Anton Karlsson, Enskede (SE); Anders Carlsson, Uppsala (SE)

(73) Assignee: CMA MICRODIALYSIS AB, Solna (SE)

(21) Appl. No.: 13/202,160

(22) PCT Filed: Feb. 17, 2010
(86) PCT No.: PCT/SE2010/050180

§ 371 Date: Oct. 31, 2011

Related U.S. Application Data
(60) Provisional application No. 61/153,453, filed on Feb. 18, 2009.

(30) Foreign Application Priority Data
Feb. 18, 2009 (SE) 0950087-7

(51) Int. Cl. A61M 25/16

(52) U.S. Cl. 604/535

(57) ABSTRACT

The present invention relates to a connector of the luer lock type having a first and a second part attached in a liquid sealing arrangement. Each part is provided with a central bore in which liquid transporting tubings are attached for establishing liquid connection in a liquid sealing arrangement between two medical devices. The tubing attached to the first part protrudes into the central bore of the second part.

### FIELD OF INVENTION

The Present invention relates to an improved connector of the luer lock type for establishing liquid connection between two parts of a medical device.

### BACKGROUND

Different techniques based on a controlled flow that interacts over a membrane with body fluids in order to study the level of substances are known. One such established technology is represented by microdialysis wherein a catheter with a semi-permeable membrane is inserted into the tissue. A low flow of perfusion fluid is pumped through the catheter for interaction with the tissue and subsequent sampling and analysis outside the body. Microdialysis has found many applications for examining and monitoring the contents fluids of extracellular spaces both preclinical, research models, and in clinical applications. Catheter probes for Microdialysis, adapted to be invasively located in the body, and systems for their operation for example disclosed in U.S. Pat. Nos. 6,264,627; 6,632,315; 6,346,090; 6,811,542; or in the Swedish patent application SE0602199-2.

More recently, a clinical demand is raised for continuous measurement of blood glucose and other key substances for efficiently monitoring critical care patients in order to more proactively being able to treat complications with life saving consequences. The two Swedish patent applications SE 081571-1 and SE 081569-5 describe microdialysis systems with adaptations to this type of on-line measurement including sensors with capacity to be incorporated in such systems. The microdialysis catheter equipment requires sterilization by radiation. However, the sensor includes components such as enzymes which are liable for this type of sterilization. For this reason, the sterilized catheter part of the microdialysis system needs to be separately attached to the sensor—carrying part of the system in the surgical procedure of connecting a microdialysis system to a patient. The coupling attachment of the sensor part needs to be performed with reliable and convenient means. A luer lock connection is a conventional and standardized connection used for safe fluid transfer with medical devices such as infusion arrangements and connections between syringes and cannulas, see for example U.S. Pat. No. 4,369,781 or U.S. Pat. No. 5,851,201. U.S. Pat. No. 5,263,945 teaches how to attach tubing in a luer coupling without using adhesives and provides a stop function for the tubing. U.S. Pat. No. 5,456,676 describes a rotatable connector for two catheters. The German Utility Model DE 8801583 describes another catheter coupling wherein a first coupling part is provided on catheter tubing threaded on a cannula sleeve which is attached to second coupling part with screw threads in order to prevent the catheter tubing from being detached.

For low flow volume systems like a microdialysis systems conventional luer lock connections can introduce a technical problem since they contribute with a hold up volume generating a delay time for the sample holding fluid to reach the sensor and introduce a sample dilution that compromises the sensor response. In other terms, the system may not have the desired clinical value if a hold up volume is introduced between the patient and the sensor. Low volume couplings are available, but generally are expensive, since they require high precision in the manufacturing process. Luer locks connectors are simple and cheap to manufacture according to standard methods. Luer lock connectors have a male and a female part with surfaces tapered according to standards, which sealingly fit together when the male part is introduced into the female part. In this type of luer connector, a small liquid filled compartment is formed between the front tip of the male part and the receiving female part that represents a hold up volume. US Patent Application published as 20080319344 discloses a luer lock coupling for connecting two tubings adapted in order to admit the two tubings to meet and thereby reduce any dead volume. This connector requires exactness in the dimensions of its luer lock parts which is a condition that is difficult to reach without very high manufacturing precision. It also requires a high tubing inner diameter and that the protruding tubing needs to be inflexible in order not to inadvertently bended. It is obvious that this solution of connecting tubings does not satisfyingly rule out the risk for stops in the flow and puts limits on the tubings to be used. It is accordingly a need to improve this type of couplings for analytical systems that operate with volumes in the scale of microliters per minute in order to avoid unreasonable delay times in the measuring equipment.

### SUMMARY OF THE INVENTION

The present invention relates to a connector of the luer lock type having a first part and a second part attached in a liquid sealing arrangement. Each part is provided with a central bore in which liquid transporting tubings are attached for establishing liquid connection in a liquid sealing arrangement between two parts of a medical device. It is characterizing for the present invention that the tubing attached to the first part of the connector protrudes into the central bore of the second part of the connector. In consequence, the hold-up volume of the connector during liquid transportation through the coupling from a proximal part to a distal part will be located in the second part of the luer type connector. Hold-up volume in this context is the volume of liquid retained by the coupling. The liquid is exemplified as flowing perfusion liquid transported from a probe with a membrane interacting with a body fluid or a body tissue to a detecting sensor.

It is an important aspect of the invention that the central bore of the second part has a contracted part with reduced diameter. A reduced diameter here has the meaning that the nominal inner diameter is of the central bore is reduced a certain predetermined distance in order provided a contracted part. The contracted part serves to establish connection between the tubing protruding from the first part into the second part and the tubing attached to the second part connected to the proximal parts of the device. Accordingly, the contracted part leaves a controlled space between the first and the second tubings which defines the hold-up volume which thereby can be controlled at a minimal level. Importantly, the contracted part also serves to separate the tubings and counteract any risk that of inadvertent contacts that cause stops in liquid connection. For this purpose, it is advantageous that the contracted part is as short as possible and preferably, its diameter is less than the outer diameter of the tubing and larger than the tubing inner diameter. It is also preferable the central bore and the tubings are dimensioned so that a thin capillary liquid film is provided around the outer peripheral area of said tubings. In other terms the diameter of the bore and the outer diameter of the tubing shall be adapted so small annular space is provided around the protruding tubing in order to employ the capillary forces in a sealing arrangement with the air column provided between the contact surfaces of the two (male and female) parts of the luer lock coupling. By such an arrangement, the coupling exerts a suitably sealing effect and the transported liquid is prevented to reach the contact surfaces and cause a leakage.

The second part of the connector, preferably a conically shaped recess connected to the central bore for receiving the tubing protruding from the first part.

Preferably, the diameter of the contracted part is less than 4 mm. Preferably, the diameter is about 0.6 mm, and most preferably about 0.1 mm. The contracted part preferably has an extension of less than 4 mm; preferably it has an extension of about 0.6 mm; and most preferably about 0.1 mm. A diameter of about 0.6 mm and extension of about 0.6 mm yields a hold-up volume of about 0.1 microliters.

A contracted part extending 0.6 mm has proven to be suitable with a tubing having an inner diameter of 0.15 mm and outer diameter of 1.0 mm. Suitably in this arrangement the central bore has a diameter of 1.1 mm.

The connector according to the present invention preferably has its second part, connected to the first parts of the device terminal sterilized by means of radiation, steam or a chemical agent. Preferably, the second part is the male part of a luer lock connector and the tubing from the female part protrudes into the bore of the male part. However, it is conceivable to let the second part be the female part of a luer lock connector and let the tubing from the male part protrude into the bore of the female part.

Advantageously, the connector serves as a coupling between a part of a medical device which would be needed to be terminally sterilized and a part of a medical device such as a sensor including liable components which cannot be terminally sterilized by conventional methods such as by radiation, steam or with chemical agents.

In one aspect of the invention, the connector is provided with liquid transporting tubings adapted to a fluid flow in the range of 0.2 to 100 microliters per minute.

The connector as described will be widely applicable in systems including liquid transportation which require a low hold-up volume. In particular, the connector is useful in systems having low flows in the microliter range, such as microdialysis systems or microfiltration systems.

In a specific embodiment, the present invention relates to a system for conducting microdialysis comprising a microdialysis probe with a microdialysis membrane for insertion into a body fluid or into a body tissue; a connector as previously described and a flow-through sensor. The connector establishes liquid connection between said microdialysis probe and said sensor.

### BRIEF DESCRIPTION OF DRAWINGS

The invention is now described, by way of example, with reference to the accompanying drawings, in which:

FIG. 1 shows schematically an assembled connector according to the present invention.

FIGS. 2a-d show the male part of the connector in more detail.

### DETAILED DESCRIPTION OF PREFERRED EMBODIMENTS

In the following a detailed description of preferred embodiments of the present invention will be given. In the drawing figures, like reference numerals designate identical or corresponding elements throughout the several figures. It will be appreciated that these figures are for illustration only and are not in any way restricting the scope of the invention. Thus, any references to direction, such as “up” or “down”, are only referring to the directions shown in the figures. Also, any dimensions etc. shown in the figures are for illustration purposes.

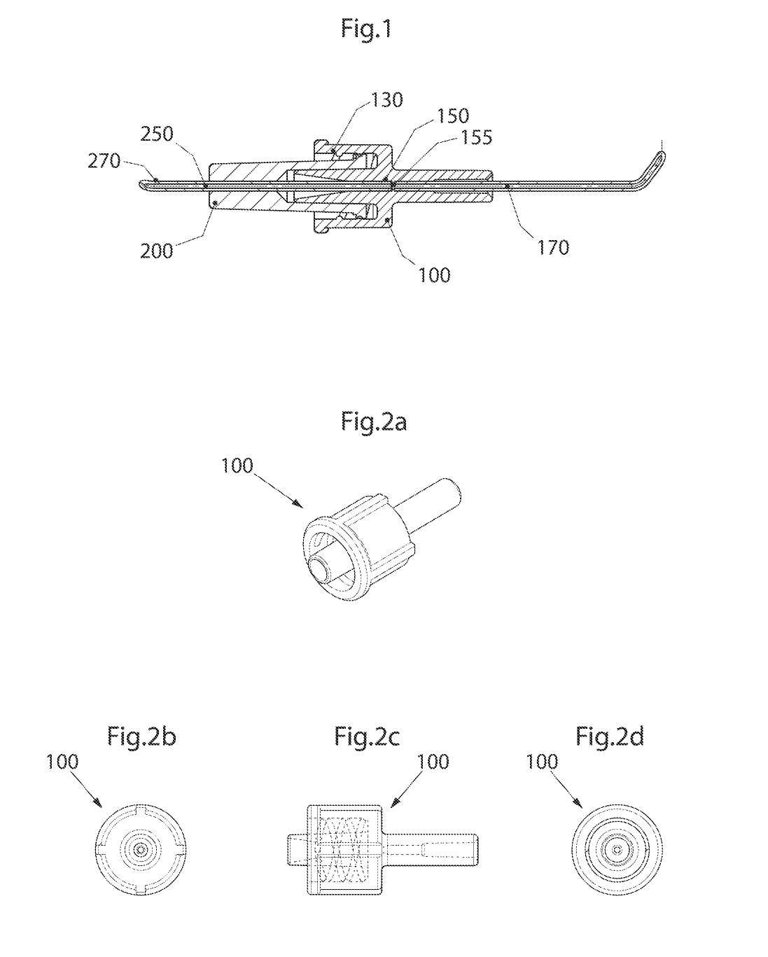

FIG. 1 shows an assembled connector according to the present invention suitable for a connecting proximal and distal parts of a microdialysis system. A male part 100 has conventional luer lock shape and is provided with a central bore 150 that has a contracted part 155 extending a certain distance. The female part 200 as attached to the male part is also conventionally shaped and provided with a central bore 250. The male part 100 has a male tubing 170 sealingly attached to the central bore 150. The male tubing is inserted in the male central bore to the end of the contracting part 155 which thereby acts as a stop. The male tubing 170 is part of the microdialysis probe (not shown) and receives dialysate therefrom. The female part has a female tubing 270 sealingly attached to the central bore 250 of the female connector part 200. The female tubing is received by the central bore 150 of the male part 100 in a sufficiently sealing manner to prevent any inadvertent flow of perfusion liquid around its circumference. The conically shape front opening 130 of the male part is designed facilitate the reception of the protruding female tubing into the male part. In the so attached connector dialysate can flow from the microdialysis probe to the sensor for detecting selected. The connector enables a convenient and reliable connection of a sterilized part of the system including the male connector part with a non-sterilized part including a sensor with liable components such as enzymes or other biomaterials. The connector arrangement provided a very low hold up volume of perfusion liquid represented by the limited and controlled distance between the two tubings spaced apart of the contracting part 155 of the bore 150 of the male part. In FIG. 1 the male and the female bore with their attached tubings are demonstrated to have the same crossection. It is of course conceivable to design and select different dimensions of the tubings and the central bore whenever relevant.

For the tubings connecting the microdialysis probe to the sensor through the connector it is advantageous that the total volume of the bore of the tubing is small so that the time needed for a certain volume of dialysate to travel from the microdialysis probe to the sensor will be low, this makes the delay in the system low. But at the same time, flow resistance should be kept low enough. For this part of the tubing one suitable dimension is a length of about 10 cm and an inner diameter of about 0.15 mm. Since the tubing is short, an inner diameter of about 0.15 mm, it does not create any problems regarding flow resistance.

The connector is made from suitable polymeric materials such as polycarbonate, polypropylene or various acrylates. The tubings can be made from polyurethanes, or polyamides. The attachment of the tubings is made by using conventional sealants, like UV curable urethane-acrylates. The choices of materials are not critical and for a skilled in the art it is conceivable to readily make suitable choices and combinations of materials to a manufacture the connector according to the present invention.

A connector as described is simple and cheap to manufacture without the need of high precision tools.

FIG. 2a shows a detailed picture of the male part of the connector 100 with suitable dimensions. The male part has a thread and cone according to the standard EN 20 594 and is depicted in a scale 2:1. FIGS. 2b and 2c are end views of the connector and FIG. 2c is side view schematically showing the thread and the central bore. Preferred embodiments of a connector according to the invention and a system including the connector have been described. A person skilled in the art realizes that these could be varied within the scope of the appended claims.

1. A connector of the luer lock type comprising a first part and a second part attached in a liquid sealing arrangement, each part being provided with a central bore in which liquid transporting tubings are attached for establishing liquid connection between two parts of a medical device, wherein the tubing attached to the first part protrudes into the central bore of the second part and the central bore of the second part has a contracted part with reduced diameter.

2. A connector according to claim 1, wherein the contacted part of the central bore of the second part is dimensioned to have a diameter less than the tubing outer diameter and larger than the tubing inner diameter.

3. A connector according to claim 1, wherein the central bore and the tubings are dimensioned so a thin capillary liquid film is provided around the outer peripheral area of said tubings.

4. A connector according to claim 1, wherein the diameter of the contracted part is less than 4 mm.

5. A connector according to claim 4, wherein the contracted part of the second part central bore extends less than 1 mm.

6. A connector according to claim 1, wherein the second part is provided with a conically shaped recess connected to the central bore for receiving the tubing protruding from the first part.

7. A connector according to claim 1, wherein only the second part has undergone terminal sterilization by means of radiation, steam or with chemical agents.

8. A connector according to claim 1, wherein the liquid transporting tubings are adapted to a fluid flow in the range of 0.2 to 100 microliters per minute.

9. A connector according to claim 1, wherein the second part is the male part of a luer lock connector, and wherein the hold-up volume during liquid transportation is located in said male part.

10. A connector according to claim 1, wherein the second part is the female part of a luer lock connector and wherein the hold-up volume during liquid transportation is located in said female part.

11. A system comprising:
a microdialysis probe with a microdialysis membrane for insertion into a body fluid or into a body tissue;
a connector according to claim 1; and
a flow-through sensor,
wherein said connector establishes liquid connection between said microdialysis probe and said sensor.

12. A connector according to claim 1, wherein the diameter of the contracted part is about 0.6 mm.

13. A connector according to claim 1, wherein the diameter of the contracted part is about 0.1 mm.

14. A connector according to claim 4, wherein the contracted part of the second part central bore extends about 0.6 mm.

15. A connector according to claim 4, wherein the contracted part of the second part central bore extends about 0.1 mm.
